(19) United States
(12) Patent Application Publication
(10) Pub. No.: US 2016/0131637 A1
(43) Pub. Date: May 12, 2016

(54) SUSPENDED NANO-ELECTRODES FOR ON-CHIP ELECTROPHYSIOLOGY

(71) Applicant: William Marsh Rice University, Houston, TX (US)

(72) Inventors: Jacob Robinson, Houston, TX (US); Daniel L. Gonzales, Houston, TX (US); Andrew Martin Bell, Houston, TX (US); Daniel Vercosa, Houston, TX (US); Benjamin Wesley Avants, Houston, TX (US)

(73) Assignee: WILLIAM MARSH RICE UNIVERSITY, Houston, TX (US)

(21) Appl. No.: 14/936,384

(22) Filed: Nov. 9, 2015

Related U.S. Application Data
(60) Provisional application No. 62/077,556, filed on Nov. 10, 2014.

(51) Int. Cl. G01N 33/487

(52) U.S. Cl. CPC G01N 33/48728

(57) ABSTRACT

A microfluidic device includes a first microfluidic channel comprising a side wall and an electrode, disposed on the side wall. The microfluidic device further includes an intersection of the first microfluidic channel and a second microfluidic channel proximate to the electrode. The electrode is suspended into an interior region of the first microfluidic chamber.

### STATEMENT REGARDING FEDERALLY SPONSORED RESEARCH OR DEVELOPMENT

The invention was made with government support under Grant Number D14AP00049 awarded by the Defense Advanced Research Projects Agency. The government has certain rights in the invention.

### BACKGROUND

In general, electrophysiology involves studying the electrical properties of biological cells. Electrophysiology is performed on different size scales from individual single ion channels up to whole organs. For example, electrical measurements of the heart may be made for diagnostic purposes.

### SUMMARY

In one aspect, a microfluidic device in accordance with one or more embodiments of the invention includes a first microfluidic channel comprising a side wall and an electrode, disposed on the side wall.

In one aspect, a method of performing an electrophysiology measurement in accordance with one or more embodiments of the invention includes positioning, using a first fluid pressure, a target at a location, the location is proximate an electrode; generating, using a second fluid pressure, direct contact between the target and the electrode; obtaining an electrophysiology measurement of the target while the target is in direct contact with the electrode; and sorting the target based on the electrophysiology measurement.

### BRIEF DESCRIPTION OF DRAWINGS

Certain embodiments of the invention will be described with reference to the accompanying drawings. However, the accompanying drawings illustrate only certain aspects or implementations of the invention by way of example and are not meant to limit the scope of the claims.

FIG. 1A shows a diagram of a system in accordance with one or more embodiments of the invention.

FIG. 1B shows a diagram of an isometric view of a microfluidic device in accordance with one or more embodiments of the invention.

FIG. 1C shows a diagram of a top view of a first example of a horizontal electrode in accordance with embodiments of the invention.

FIG. 1D shows a diagram of a top side of a first example of a horizontal electrode in accordance with embodiments of the invention.

FIG. 1E shows a diagram of a top view of a second example of a horizontal electrode in accordance with embodiments of the invention.

FIG. 1F shows a diagram of a side view of a second example of a horizontal electrode in accordance with embodiments of the invention.

FIG. 1G shows a diagram of a top view of a third example of a horizontal electrode in accordance with embodiments of the invention.

FIG. 1H shows a diagram of a side view of a third example of a horizontal electrode in accordance with embodiments of the invention.

FIG. 1I shows a diagram of a top view of a fourth example of a horizontal electrode in accordance with embodiments of the invention.

FIG. 1J shows a diagram of a side view of a fourth example of a horizontal electrode in accordance with embodiments of the invention.

FIG. 1K shows a diagram of a top view of a fifth example of a horizontal electrode in accordance with embodiments of the invention.

FIG. 1L shows a diagram of a side view of a fifth example of a horizontal electrode in accordance with embodiments of the invention.

FIG. 1M shows a diagram of a top view of a sixth example of a horizontal electrode in accordance with embodiments of the invention.

FIG. 1N shows a diagram of a side view of a sixth example of a horizontal electrode in accordance with embodiments of the invention.

FIG. 1O shows a method of forming a microfluidic device in accordance with embodiments of the invention.

FIG. 2A shows a diagram of a system in accordance with embodiments of the invention.

FIG. 2B shows a method of operating a system in accordance with embodiments of the invention.

FIG. 3A shows a diagram of an example operation of a system in accordance with embodiments of the invention.

FIG. 3B shows a diagram of a second example operation of a system in accordance with embodiments of the invention.

FIG. 3C shows a diagram of a third example operation of a system in accordance with embodiments of the invention.

FIG. 3D shows a diagram of a fourth example operation of a system in accordance with embodiments of the invention.

FIG. 4A shows a microscopy image of a microfluidic device in accordance with embodiments of the invention.

FIG. 4B shows a second microscopy image of a microfluidic device in accordance with embodiments of the invention.

FIG. 4C shows an example of an electrophysiology measurement in accordance with embodiments of the invention.

FIG. 4D shows a first example of a phenotypic map in accordance with embodiments of the invention.

FIG. 4E shows a second example of a phenotypic map in accordance with embodiments of the invention.

FIG. 4F shows a third example of a phenotypic map in accordance with embodiments of the invention.

FIG. 4G shows a fourth example of a phenotypic map in accordance with embodiments of the invention.

FIG. 4H shows an example of a legend of a phenotypic map.

FIG. 5A shows a fifth example of a phenotypic map in accordance with embodiments of the invention.

FIG. 5B shows a sixth example of a phenotypic map in accordance with embodiments of the invention.

### DETAILED DESCRIPTION

Specific embodiments will now be described with reference to the accompanying figures. In the following description, numerous details are set forth as examples of the invention. It will be understood by those skilled in the art that one or more embodiments of the present invention may be practiced without these specific details and that numerous variations or modifications may be possible without departing from the scope of the invention. Certain details known to those of ordinary skill in the art are omitted to avoid obscuring the description.

Embodiments of the invention relate to systems for performing electrophysiology measurements. Electrophysiology measurements are measurements of electrical potential or electrical currents across cell membranes. In some measurements, the cell membrane is ruptured or penetrated to record intracellular electrical potentials or currents.

A system in accordance with embodiments of the invention may position a target with respect to an electrode by applying fluid pressures to the target. Once positioned, the system may perform an electrophysiology measurement on the target through the electrode. After performing the electrophysiology measurement, the target may apply fluid pressures to the target to move the target to a sorting area. The system may then sort the target based on the result of the electrophysiology measurement.

The system may include a microfluidic device that includes channels for positioning the target with a respect to an electrode disposed on a side wall of one of the channels. For example, a channel may be filled with a fluid and the target. Applying fluid pressure, by the system, to one end of the channel may cause the fluid within the channel to flow along the channel in a direction corresponding to the applied pressure. Flowing the fluid within the channel may move the target in the direction of the fluid flow.

More specifically, the microfluidic device may include a first channel with an electrode disposed on a side wall of the first channel. A first fluid pressure may be applied to the first channel, by the system, that positions the target along the length of the first channel corresponding to a location of the electrode.

The microfluidic device may also include a second channel that intersects with the first channel near the location of the electrode. In one or more embodiments of the disclosure, the intersection may be in the form of a three way junction, e.g. a T junction or a Y junction. In one or more embodiments of the disclosure, the intersection may be in the form of a four way junction, e.g. a crossroads junction. A second fluid pressure may be applied, by the system, to the second channel to press the target against the electrode disposed on the sidewall of the microfluidic device when the target is at a location corresponding to the location of the electrode. In one or more embodiments of the disclosure, pressing the target against the electrode may cause the target to deform around the electrode which may maximize the surface area contact between the target and the electrode. In one or more embodiments of the disclosure, the electrode may penetrate the target when the target is pressed against the electrode.

The electrode may be a suspended nano-electrode formed by a lithographic process. The electrode may be a direct contact electrode that forms an electrical connection with a target when the target is in contact with the electrode.

A first portion of the electrode may extend into the first channel and a second portion may extend into the side wall of the first channel and may connect to additional electrical components of the system such as amplifiers or other devices that may support performance of electrophysiology measurements of the target. The system may include additional components, such as a measurement controller, that performs electrophysiology measurements on targets by applying voltage or current to the electrode.

Once an electrophysiology measurement is performed, the second pressure may be changed, by the system, to separate or disengage the target from the electrode. After separation from the electrode, the first pressure may be changed, by the system, to move the target to a sorting area connected to the first channel.

The system may include a sorting control unit that sorts the target in the sorting area based on the result of the electrophysiology measurement. For example, the sorting area may include a three-way junction that is connected to three separate sorting tanks. Based on the outcome of the electrophysiology measurement, the target may be sorted to one of the sorting tanks by applying a sorting fluid pressure. For example, if the target is to be sorted to a first sorting tank based on the result of the electrophysiology measurement, a negative fluid pressure may be applied to the first sorting tank which may cause the target to flow into the first sorting tank through a first sorting channel.

The system may also include an inspection system that identifies targets as the targets traverse the microfluidic device and may provide the location of the target to other components of the device. For example, the inspection system may be a camera system that obtains images through a top, transparent surface of the device. The inspection system may identify the target and the location of the target based on the images. The inspection system may provide the location of the target to a system controller of the system that orchestrates the positioning of the target within the microfluidic device. By providing the location of the target to the system controller, the system may apply fluid pressures within the microfluidic device to move the target.

The target may be a small organism, a single cell, microbe, vesicle, organelle, protein assembly, or smaller structures such as individual proteins. The system may sort targets in an efficient and automated manner by receiving targets from a target supply, positioning the targets with respect to an electrode, pressing the targets against the electrode, performing an electrophysiology measurement, and sorting the targets based on the electrophysiology measurement.

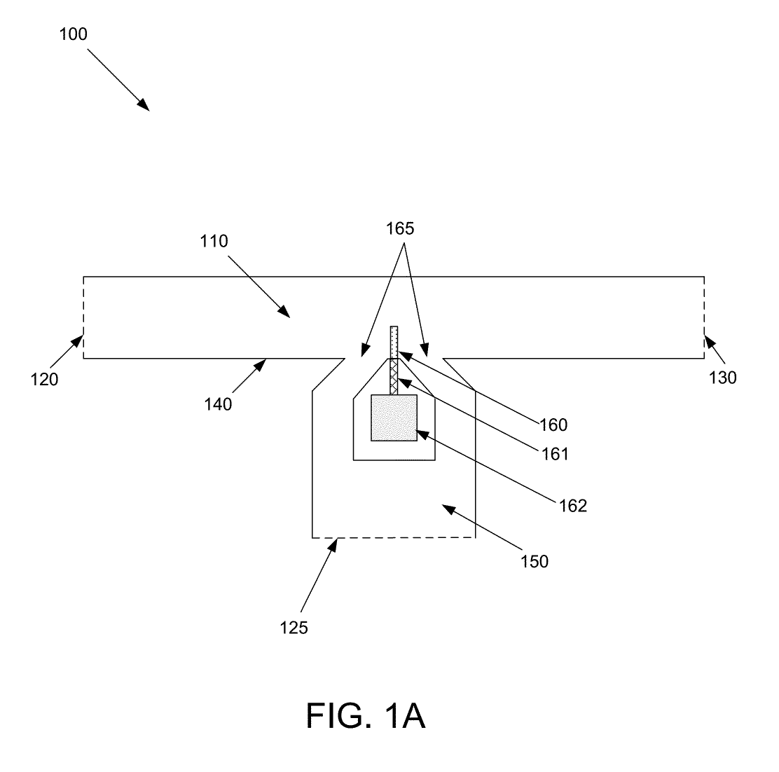

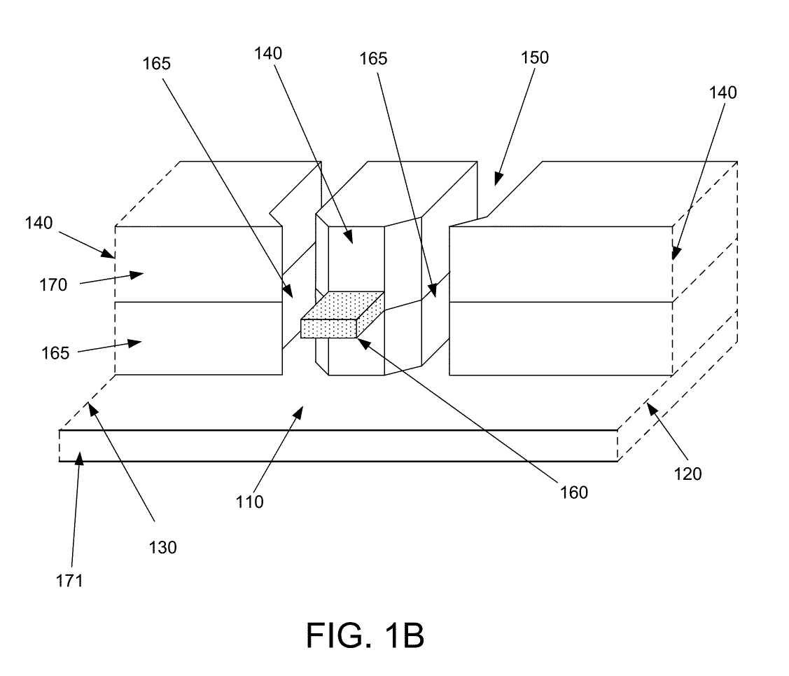

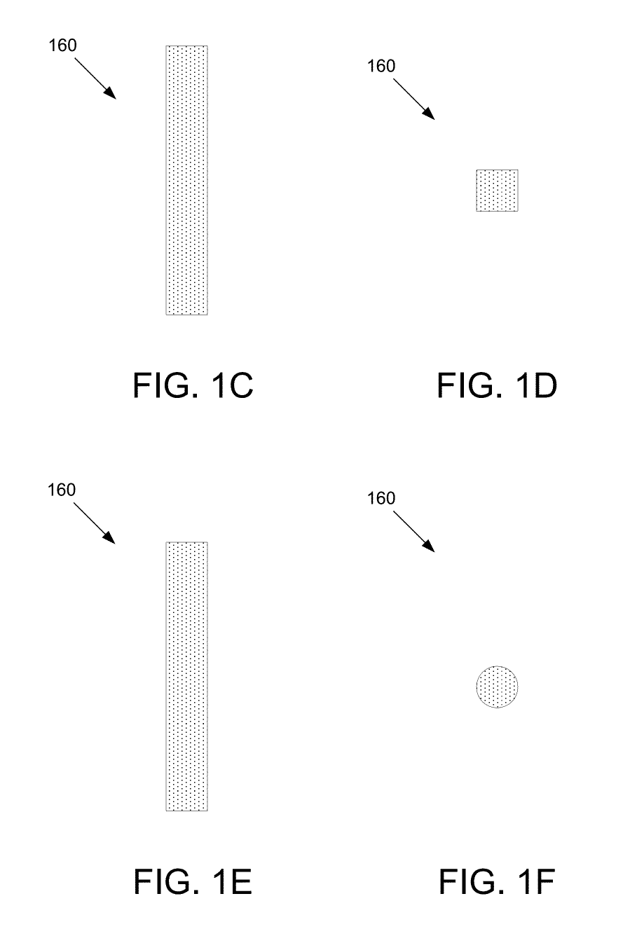

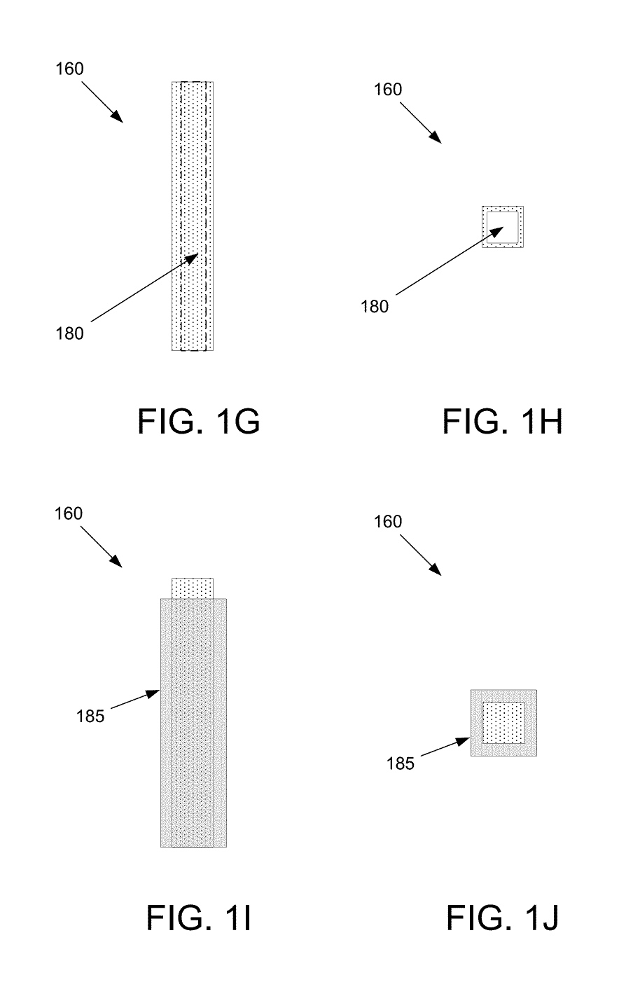

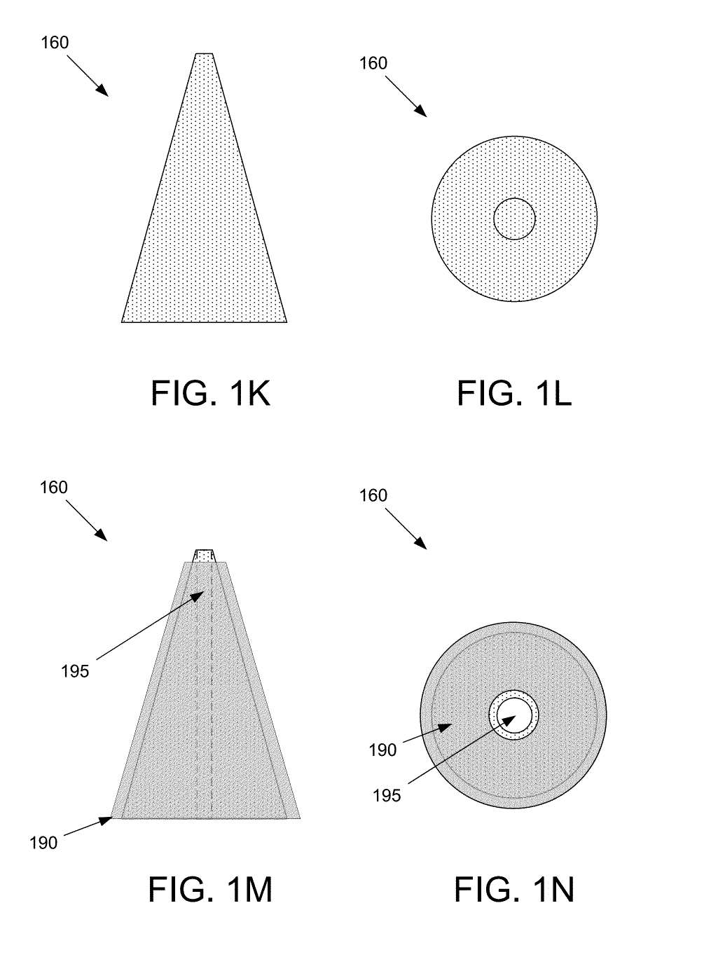

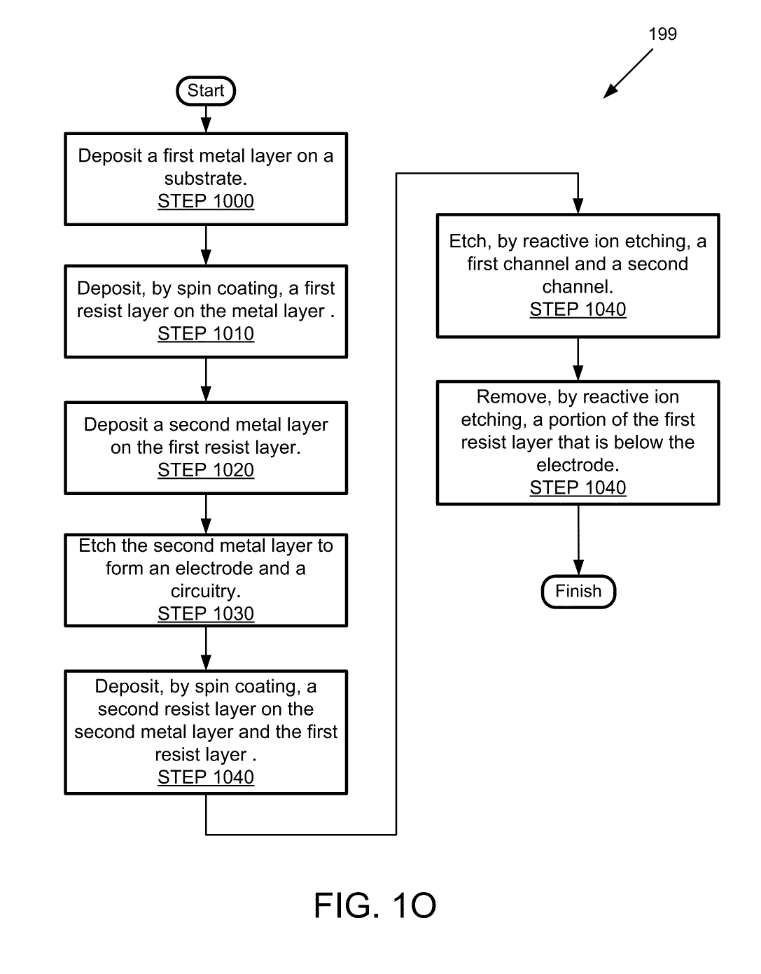

FIG. 1A shows a diagram of a microfluidic device (100) in accordance with one or more embodiments of the invention. The microfluidic device (100) may include a first channel (110) that includes a first port (120) and a second port (130). The first channel maybe a microfluidic channel for fluid flow. The first channel may be rectangular in cross section and include two sidewalls, a top, and a bottom. Each port may be on a first end and second end of the first channel (110), respectively. Fluids may flow into or out of the first port (120) and second port (130).

The microfluidic device (100) may include a second channel (150) that intersects with the first channel (110). The second channel may include a third port (125) that connects to other components of the device (100). Fluids may flow into or out of the third port (125).

The microfluidic device (100) may include an electrode (160), disposed on a sidewall (140) of the first channel (110). In one or more embodiments of the disclosure, the electrode (160) may be proximate to the intersection of the first channel (110) and the second channel (150).

The intersection between the first channel (110) and the second channel (150) may contain one or more openings that allows fluids to flow into the first channel (110) from the second channel (150) and fluids to flow into the second channel (150) from the first channel (110). The intersection of the first channel (110) and second channel (150) is drawn as a pair of sub channels (165). However, any number and arrangement of sub-channels between the first channel (110) and second channel (165) that may be used to control fluid flow at the intersection of the first channel (110) and second channel (150) without departing from the scope of the disclosure. The direction of fluid flow between the channels may depend on the fluid flows into or out of the first port (120), the second port (130), and/or the third port (125). The microfluidic device (100) may also include wiring (161) and electronics (162). The wiring (161) and electronics (162) may support performance of electrophysiology measurements.

While the microfluidic device (100) has been shown in FIG. 1A as including a single intersection, the microfluidic device (100) may include a number of intersections between the first channel (100) and a number of additional channels (not shown) without departing from the scope of the disclosure. Each intersection may include an additional electrode (not shown) and associated electronics as described above. By including a number of intersections, a number of targets may characterized in parallel. For example, a continuous flow of a fluid containing targets may flow along the first channel (110). As targets pass a first intersection, the system may apply a fluid pressure to the first intersection to trap or capture a target. When a target is trapped or captured at the first intersection, the fluid may continue to flow past the first intersection, along the first channel (100), which may supply targets to additional intersections downstream of the first intersection. Thus, embodiments of the disclosure may enable a number of targets to be trapped or captured and characterized corresponding to the number of intersections along the single channel (100) at any one time.

FIG. 1B shows an isometric view of a cut view along the length of the first channel (110) and electrode (160) in accordance with one or more embodiments of the disclosure. The sidewalls of the microfluidic device include a first layer (165) and a second layer (170), which are described below. In one or more embodiments of the invention, the first layer (165) and second layer (170) are resist materials or substrate materials. The first layer (165) of the sidewalls may be disposed on a bottom substrate (171). A top substrate (not shown) may be disposed on the second layer (170). In one or more embodiments of the disclosure, the top substrate is a transparent material such as glass.

As seen in FIG. 1B, the electrode (160) may extend from the side wall of the first channel (110). While the electrode (160) is drawn as an extruded rectangular cross section, numerous variations are possible. FIGS. 1C-1N illustrate various embodiments of the electrode (160). Forming the electrode (160) as a horizontal, suspended structure may greatly reduce the manufacturing complexity of the device. Further, suspending the electrode may allow targets to deform around the electrode which may improve the magnitude of the signal received during electrophysiology measurements. Additionally, the suspended electrode may penetrate the target by mechanical force, electrical pulses, or chemical surface modification of the electrode. Measurements within the target may provide larger magnitudes of signals received during electrophysiology measurements. Lastly, a horizontal structure may be easily fabricated via lithographic methods which enables high precision of features and scalability of manufacturing.

FIGS. 1C and 1D shows a top down and cross sectional view of an electrode (160), respectively, in accordance with one or more embodiments of the disclosure. As seen from FIGS. 1C and 1D, the electrode (160) may be an extruded square.

FIGS. 1E and 1F shows a top down and cross sectional view of an electrode (160), respectively, in accordance with one or more embodiments of the disclosure. As seen from FIGS. 1E and 1F, the electrode (160) may be an extruded circle.

FIGS. 1G and 1H shows a top down and cross sectional view of an electrode (160), respectively, in accordance with one or more embodiments of the disclosure. As seen from FIGS. 1G and 1H, the electrode (160) may be a extruded square, rectangle, or circle that includes a hollow portion (180). A hollow portion (180) may allow for chemical delivery to the target and move electrochemical reactions that may occur as part of an electrophysiology measurement away from the target. By moving electrochemical reactions away from the target, the target may be preserved. In some cases, electrophysiology measurements may change, for example, the ion concentration at the point intersection between a target and a solid electrode. Large local ion concentrations may change the target. A hollow portion (180) of an electrode (160) may move the location of ion generation away from the target and therein prevent change of the target. By preventing ion concentration buildup, embodiments of the disclosure may enable electrophysiology measurements using more benign voltage and current clamp recordings that require faradaic currents.

FIGS. 1I and 1J shows a top down and cross sectional view of an electrode (160), respectively, in accordance with one or more embodiments of the disclosure. As seen from FIGS. 1I and 1J, the electrode (160) may be a extruded square, rectangle, or circle that includes a dielectric layer (185). The dielectric layer (185) may improve a seal between the target and the electrode (160) when the target is pressed against the electrode. The dielectric layer (185) may also improve the quality of the electrophysiology measurement by reducing the portion of the metal electrode (160) that is exposed to fluids in the first channel (110).

FIGS. 1K and 1L shows a top down and cross sectional view of an electrode (160), respectively, in accordance with one or more embodiments of the disclosure. As seen from FIGS. 1K and 1L, the electrode (160) may be a cone that narrows from a circle of a first radius to a circle of a second radius where the second radius is smaller than the first radius. The larger radius circle may be connected to the side wall of the first channel (110) and the smaller radius may protrude into the first channel (110), or the reverse. The target may more easily deform to the outer profile of the cone shaped electrode (160) and therein create a superior seal between the target and the electrode (160). Creating a superior seal between a target and the electrode (160) may improve the quality an electrophysiology measurement.

FIGS. 1M and 1N shows a top down and cross sectional view of an electrode (160), respectively, in accordance with one or more embodiments of the disclosure. As seen from FIGS. 1K and 1L, the electrode (160) may be a cone that narrows from a circle of a first radius to a circle of a second radius where the second radius is smaller than the first radius. The electrode may include a dielectric layer (190), disposed on the outside of the electrode, that covers a majority of the electrode (160). The electrode may also include a hollow portion (195), disposed within the electrode. The larger radius circle may be connected to the side wall of the first channel (110) and the smaller radius may protrude into the first channel (110), or the reverse.

FIG. 1O shows a method according to one or more embodiments of the invention. The method depicted in FIG. 1O may be used to produce a microfluidic device (100) in accordance with one or more embodiments of the invention. One or more steps shown in FIG. 1O may be omitted, repeated, and/or performed in a different order among different embodiments of the invention.

At STEP 1000, a first metal layer is disposed on a substrate. In one or more embodiments of the disclosure, the substrate may be a silicon wafer, glass sheet, or other rigid material. In one or more embodiments of the disclosure, the substrate may be a polymer film covered hard substrate such as a silicon wafer, glass sheet, or other rigid material. In one or more embodiments of the disclosure, the first metal layer may be deposited onto the substrate by electron beam evaporation. The first metal layer may be deposited and/or formed using techniques other than electron beam evaporation without departing from the invention. In one or more embodiments of the disclosure, the first metal layer may promote adhesion between the substrate and subsequently deposited layers.

At STEP 1010, a first resist layer is deposited onto the first metal layer. The first resist layer may form the first layer (165, FIG. 1B). The first resist layer may be deposited by spin coating, spray coating, or any other method as would be known to one of ordinary skill in the art.

At STEP 1020, a second metal layer is deposited onto the first resist layer. In one or more embodiments of the disclosure, the second metal layer may be platinum. In one or more embodiments of the disclosure, the second metal layer may be 60 nm thick. In one or more embodiments of the disclosure, the second metal layer may deposited by sputtering or metal vapor deposition. The second metal layer may be deposited and/or formed using techniques other than sputtering or metal vapor deposition without departing from the invention.

At STEP 1030, the second metal layer is etched to form an electrode and additional circuitry. Etching may include depositing a resist layer onto the second metal layer, imaging of the resist layer, and then performing a lift off process that removes a portion of the second metal layer. Removing a portion of the second metal layer may create the electrode and the additional circuitry. A portion of the second metal layer remaining after etching may extend over a portion of the first resist layer that will be removed to suspend the portion of the second metal layer. One of ordinary skill in the art will appreciate that the electrode and additional circuitry may be formed using other techniques without departing from the invention.

At STEP 1040, a second resist layer is deposited onto the second metal layer and an exposed portion the first resist layer. In one or more embodiments of the disclosure, the second resist layer may form the second layer (170, FIG. 1B). The second resist layer may be deposited by, for example, spin coating or spray deposition.

At STEP 1050, the first channel and second channel are formed by reactive ion etching. In one or more embodiments of the disclosure, reactive ion etching removes a portion of the second resist layer and the first resist layer corresponding the location of the first channel (110) and the second channel (150). One of ordinary skill in the art will appreciate that techniques other than reactive ion etching may be used to remove the portion of the first and/or second resist layers without departing from the invention.

At STEP 1060, the electrode is suspended by reactive ion etching. In one or more embodiments of the disclosure, reactive ion etching removes a portion of the first resist layer disposed between the substrate (171) and the electrode (160). One of ordinary skill in the art will appreciate that techniques other than reactive ion etching may be used to remove the first resist layer without departing from the invention.

After performing the steps as discussed above, a transparent substrate such as a glass or plastic sheet may be disposed on top of the second resist layer as a top of the device. Thus, the method shown in FIG. 1O may be used to produce the microfluidic device (100) shown in FIG. 1A.

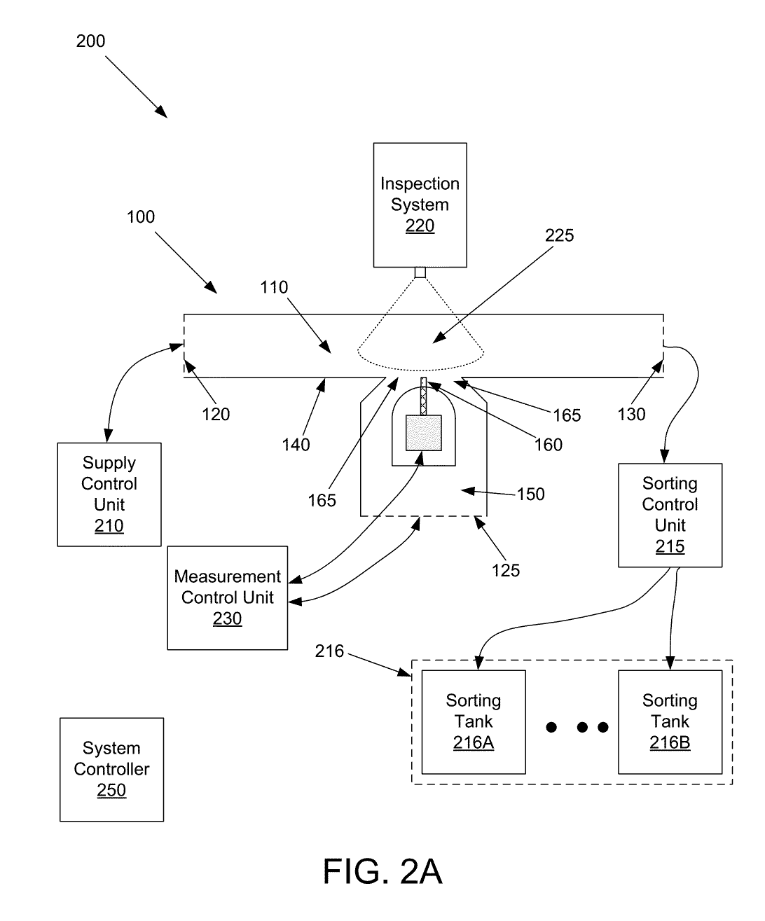

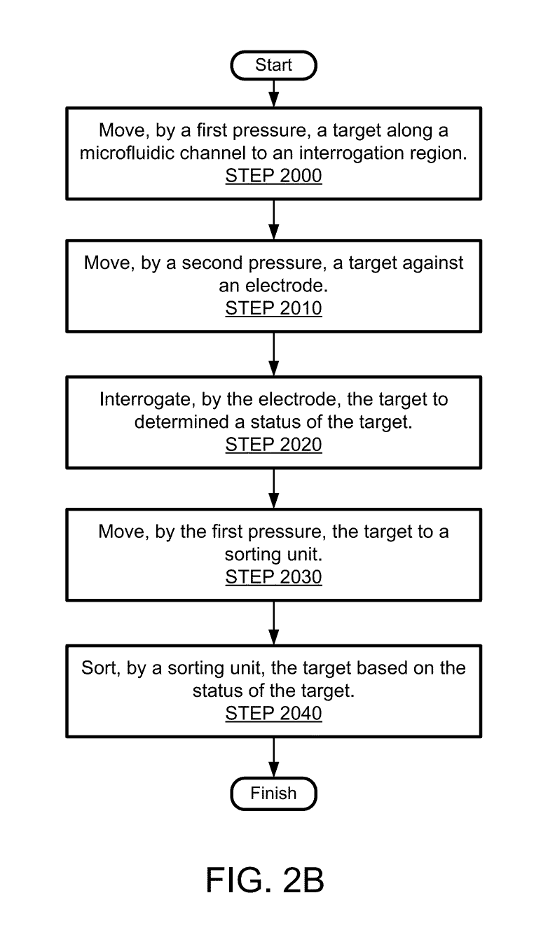

FIG. 2A shows a system (200) in accordance with one or more embodiments of the disclosure. The system includes the microfluidic device (100) and a number of other components. The operation of the system may be controlled by a system controller (250) that communicates with the other components of the system by a network (not shown).

The system (200) may include a supply control unit (210). The supply control unit (210) may be connected to the first port (120) by a fluid connection as indicated by the double ended arrow. The supply control unit (200) may include a fluid supply containing targets. For example, the fluid supply may contain a liquid that includes cells. The supply control unit (200) may include a pump that pumps fluid to or from the fluid supply. The supply control unit (200) may pump a fluid out of the fluid supply, through the first port (120), and into the first channel (110) as indicated by the arrow from the supply control unit (200) to the first port (120). By pumping the fluid into the first channel (110), the supply control unit (200) may generate a pressure in the first channel (110) that causes fluid in the first channel (110) to flow toward the second port (130). By causing fluid flow within the first channel (110), the supply control unit (200) may position targets along the length of the first channel (110).

The pump may also pump fluids out of the first channel (110), through the first port (120), and into the fluid supply. By pumping the fluid into the fluid supply, the supply control unit (200) may generate a pressure in the first channel (110) that causes fluid in the first channel (110) to flow toward the first port (120). By causing fluid flow within the first channel (110), the supply control unit (200) may position targets along the length of the first channel (110).

The supply control unit (210) may include a communication unit (not shown) to communicate with the system controller (250) and other components of the system (200). The supply control unit (210) may send and receive messages by the communication unit.

The system (200) may include a sorting control unit (210). The sorting control unit (210) sorts targets based on an electrophysiology measurement. The sorting control unit (210) may receive targets from the first channel (110) by the second port (130). Once received, the sorting control unit (215) may sort the target into a tank (216) based on the result of an electrophysiology measurement.

The sorting control unit (215) may include a communication unit (not shown) to communicate with the system controller (250) and other components of the system (200). The sorting control unit (215) may send and receive messages by the communication unit.

The system (200) may include a measurement control unit (230). The measurement control unit (230) may be connected to the circuitry (162, FIG. 1A), wiring (161, FIG. 1A), and electrode (160) as indicated by the double sided arrow. The measurement control unit may perform an electrophysiology measurement, as is known in the art, when the target is pressed against the electrode (160). The measurement control unit (230) may also include a pump that pumps fluids into or out of the third port (125). By pumping fluids into our out of the third port (125), the measurement control unit (230) may cause a pressure in the second channel (150) that causes a fluid flow that may press a target against the electrode (160) or push the target away from the electrode (160).

The measurement control unit (230) may include a communication unit (not shown) to communicate with the system controller (250) and other components of the system (200). The measurement control unit (230) may send and receive messages by the communication unit.

The system (200) may include an inspection system (220). The inspection system (220) determines the location of targets within the microfluidic device (100). The inspection system (220) may include a camera that images targets within the microfluidic device (100) through the top substrate of the microfluidic device (100). As noted above, the top substrate may be transparent. The inspection system (220) may determine the location of targets within the system (200) and provide the location information to other components of the system (200).

The inspection system (220) may include a communication unit (not shown) to communicate with the system controller (250) and other components of the system (200). The inspection system (220) may send and receive messages by the communication unit.

The system (200) may include a system controller (250). The system controller (250) communicates with and controls the operation of the supply control unit (210), inspection system (220), measurement control unit (230), and the sorting unit (215). The system controller is configured to direct the operations of the aforementioned system components to position targets within the microfluidic device (100), perform an electrophysiology measurement on the target, and sort the target based on the result of the electrophysiology measurement. The system controller (250) may include a processor and computer instructions stored on a non-transitory computer readable media.

The system controller (250) may include a communication unit (not shown) to communicate with other components of the system (200). The system controller (250) may send and receive messages by the communication unit.

FIG. 2B shows a method according to one or more embodiments of the invention. The method depicted in FIG. 2B may be used to perform an electrophysiology measurement in accordance with one or more embodiments of the invention. One or more steps shown in FIG. 2B may be omitted, repeated, and/or performed in a different order among different embodiments of the invention.

At STEP 2000, a target is moved along a first channel of a microfluidic device by a first pressure. The target may be moved to a location corresponding to a location of an electrode. In one or more embodiments of the disclosure, moving the target along the first channel positions the target proximate to an electrode disposed on a wall of the first channel. In one or more embodiments of the disclosure, the first pressure may be applied by a supply control unit.

At STEP 2010, the target is pressed against the electrode by a second pressure in response to the target reaching a location corresponding to an electrode. In one or more embodiments of the disclosure, the second pressure is applied to the target by a fluid flow into a second channel that intersects the first channel proximate to the electrode. In one or more embodiments of the disclosure, the second pressure may be applied by a measurement control unit.

At STEP 2020, the target is interrogated by an electric signal applied by the electrode in response to the target being pressed against the electrode. In one or more embodiments of the disclosure, a status of the target is determined based on the interrogation. In one or more embodiments of the disclosure, the interrogation may be applied by a measurement control unit.

At STEP 2030, the target is moved by the first pressure to a sorting area in response to an interrogation. In one or more embodiments of the disclosure, the first pressure may be applied by a supply control unit.

At STEP 2040, the target is sorted based on the status of the target in response to the target moving to the sorting area. For example, genetically-encoded voltage sensitive fluorescent indicators (GEVIs) may be sorted based on how quickly the florescence intensity changes in response to a change in the membrane potential. Mutant variants of GEVIs that respond more quickly than known GEVIs may then be separated from slower variants. In another example, cells may be sorted based on response to a chemical reagent. Neurons, for example, may be included as targets in a fluid with an elevated level of carbon dioxide. Some of the targets may depolarize in response to the elevated level of carbon dioxide. The depolarization of some of the targets may be measured by the electrophysiology measurements according to embodiments of the disclosure. The targets may then be sorted based on sensitivity to carbon dioxide by the electrophysiology measurement

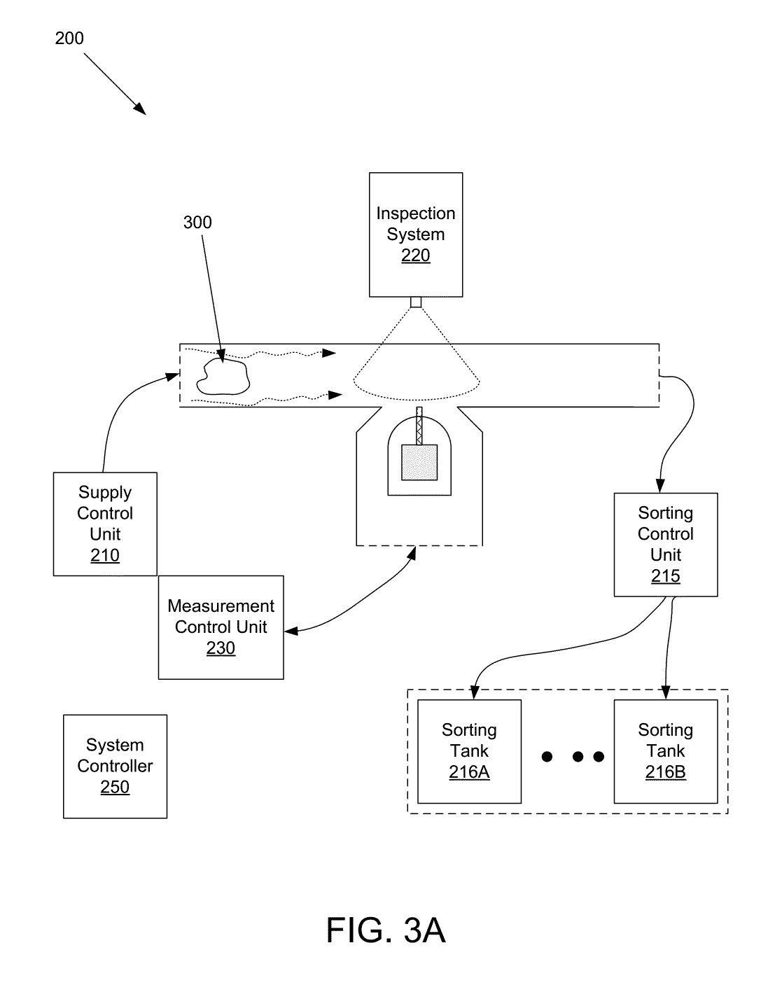

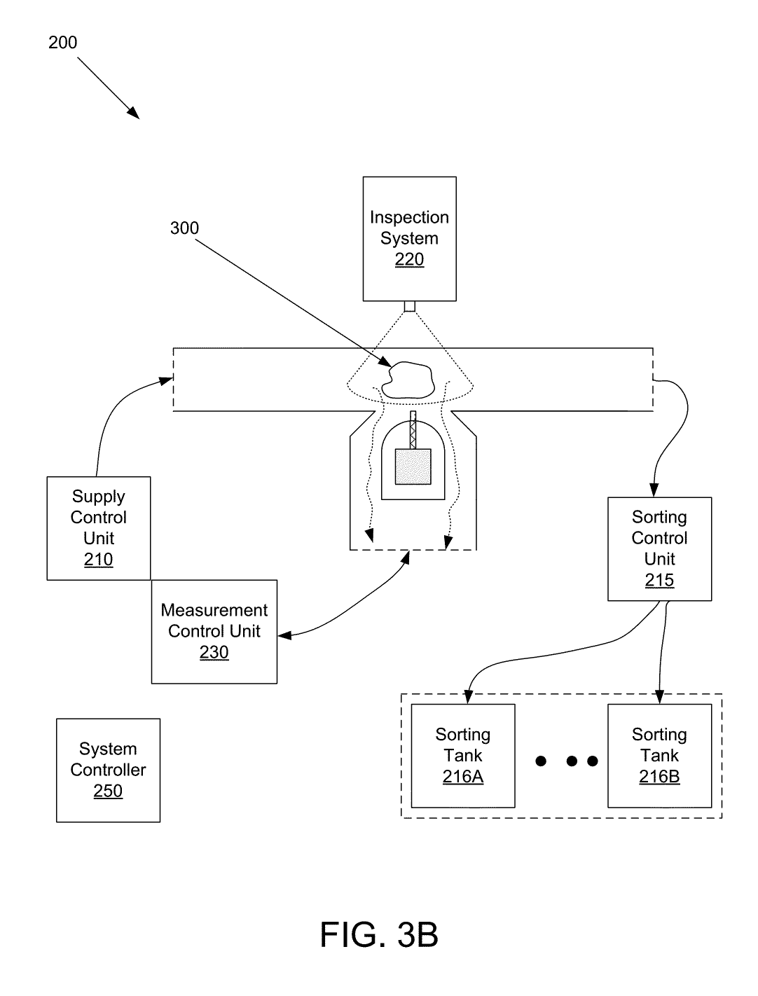

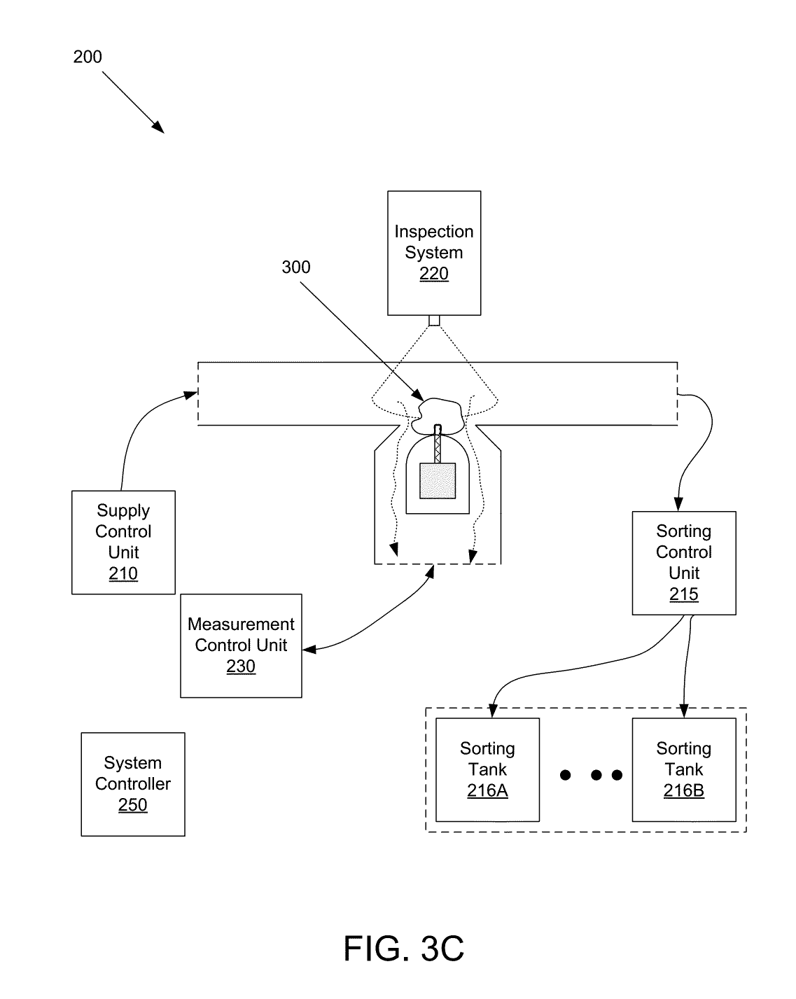

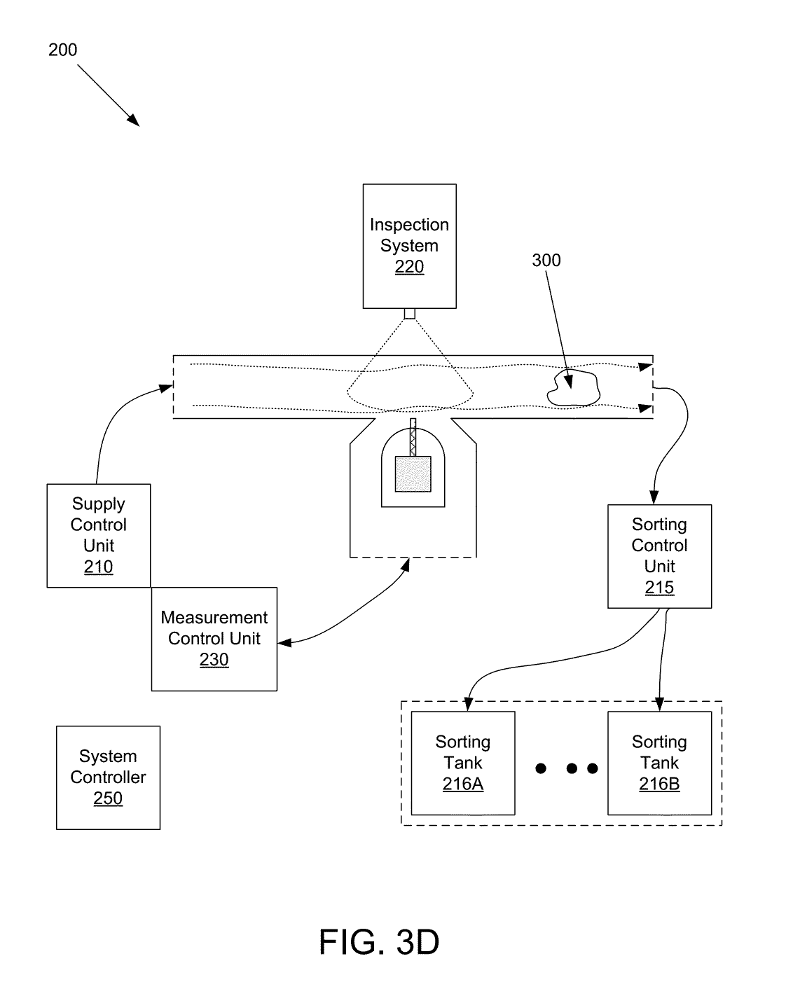

FIGS. 3A-3D show an example of sorting a target according to the method of FIG. 2B and system of FIG. 2A. In FIG. 3A, a target (300) is located in the first channel (110). The inspection system (220), supplies location information of the target to the system controller (250). In response to receiving the location information from the inspection system (220), the system controller (250) identifies that the target (300) is not in position to be measured. In response to determining that the target (300) is not in position to be measured, the system controller (250) sends a signal to supply control unit (210) indicating that the target needs to be moved along the first channel (110). In response to receiving the signal, the supply control unit (210) activates the supply control unit pump to create a pressure in the first channel (110). The created pressure causes a fluid flow that moves the target (300). The fluid flow in this example is indicated as arrows with dashed tails.

In FIG. 3B, the fluid flow caused by the supply control unit (210) moved the target (300) along the length of the first channel (110). During the movement of the target (300), the inspection system (220) monitores the location of the target (300) and sent the location of the target (300) to the system controller (250). The system controller (250) determines, based on the location of the target provided by the inspection system (220), that the target (300) reached a location within the first channel (110) corresponding to the location of the electrode (160). In response to determining that the target (300) reached the corresponding location, the system controller (250) sends a signal to the supply control unit (210) to stop the pump. By stopping the pump, the flow along the first channel (110) was stopped which stopped the movement of the target (300) along the length of the first channel (110). The system controller (250), in response to determining that the target (300) reached the corresponding location, also sent a signal to the measurement control unit indicating the target (300) was at the corresponding location. In response to receiving the message, the measurement control unit (230) activates a pump that creates a fluid flow along the second channel (150), indicated by arrows with dotted tails.

In FIG. 3C, the fluid flow along the second channel (150) pressed the target (300) against the electrode (160). During the pressing of the target (300), the inspection system (220) monitors the location of the target (300) and sends the location of the target (300) to the system controller (250). The system controller (250) determines, based on the location of the target provided by the inspection system (220), that the target (300) reaches a location corresponding to a measurement location. In response to determining that the target (300) reaches the measurement location, the system controller (250) sends a signal to the measurement control unit (230) indicating that the target (300) is in a measurement location. The measurement control unit (230) initiates an electrophysiology measurement and determines a status of the target (300) in response to the received signal.

In FIG. 3D, the measurement control unit (230) sends a signal to the system controller (250) indicating the status of the target (300). In response to receiving the signal indicating the status of the target, the system controller (250) sends a message to the supply control unit (210) indicating that the pump should be activated. In response to receiving the pump activation signal, the supply control unit activates a pump that causes a fluid flow in the first channel (110), as indicated by arrows with dotted tails. The fluid flow moves the target (300) to a sorting control unit (215). In response to the sorting control unit (215) receiving the target (300), the system controller (250) sends the sorting control unit (215) a message indicating the status of the target (300). In response to receiving the signal indicating the status of the target (300), the sorting control unit (215) sorts the target (300) to a tank (216) based on the indicated status of the target (300).

Thus, the method and system shown in FIGS. 3A-3D may be used to sort targets based on electrophysiological measurements in an automatic manner or semi-automatic manner with little to no user intervention.

The following are examples of usages and/or microfluidic devices in accordance with one or more embodiments of the invention. The following examples are explanatory examples and not intended to limit the invention.

### EXAMPLE 1

In the following example, a microfluidic device including a horizontal electrode is utilized to diagnose gene expressions that may lead to the pathogenesis of amyotrophic lateral sclerosis (ALS) or Parkinsons's disease (PD) in animal models.

Three varieties of Caenorhabditis elegans (“C. elegans”) nematode worms were selected for characterization using a microfluidic device in accordance with embodiments of the invention. The first variety of C. elegans had expressions of human genes know to be implicated in the pathogenesis of ALS. Specifically, the first variety expressed G85R Cu, Zn-superoxide dismutase-1 (SOD1). The second variety of C. elegans had expressions of human genes known to be implicated in the pathogenesis of PD. More specifically, the second variety expressed α-synuclein. The third variety of C. elegans was a wild type (WT) used as a control.

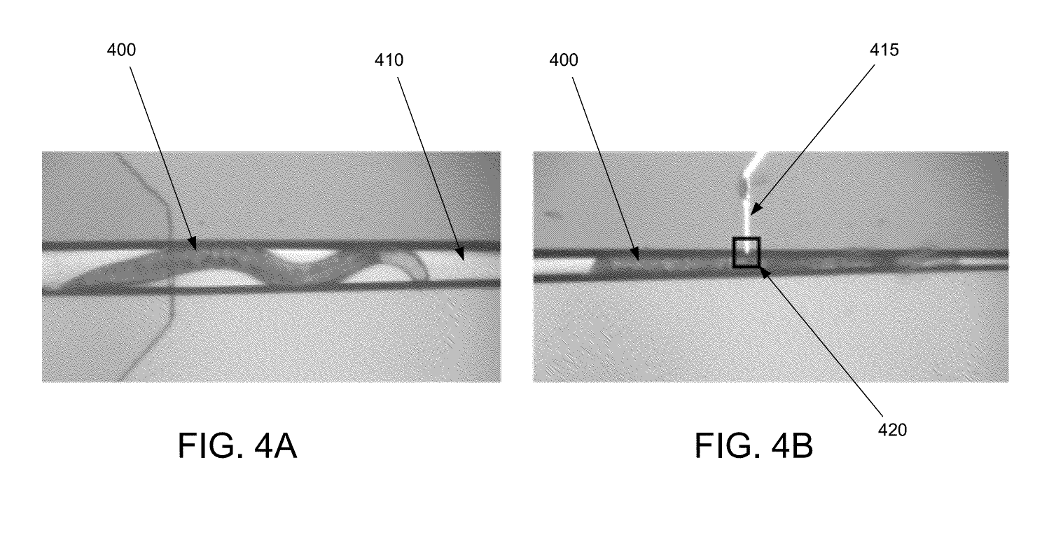

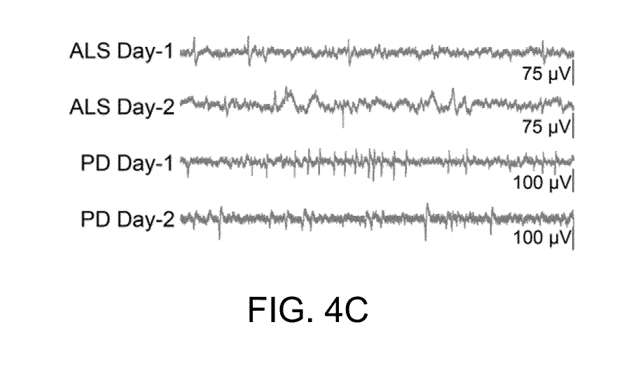

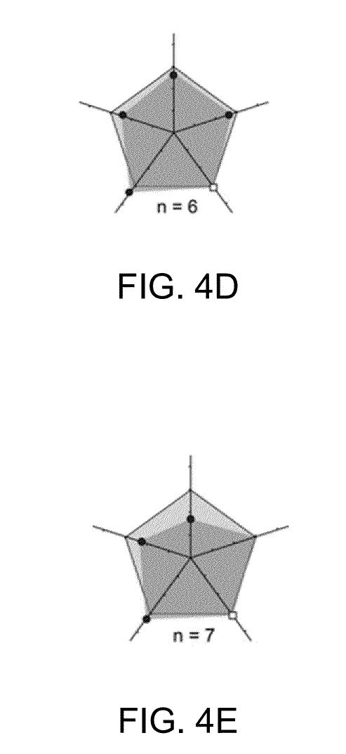

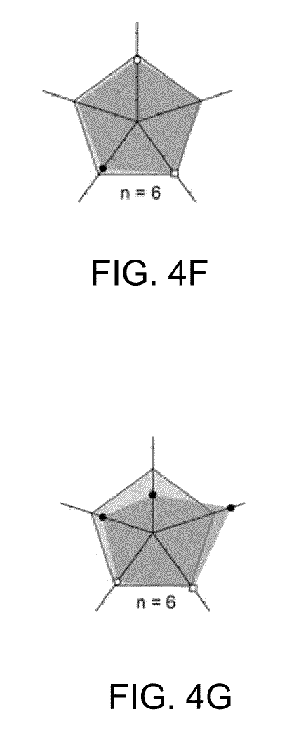

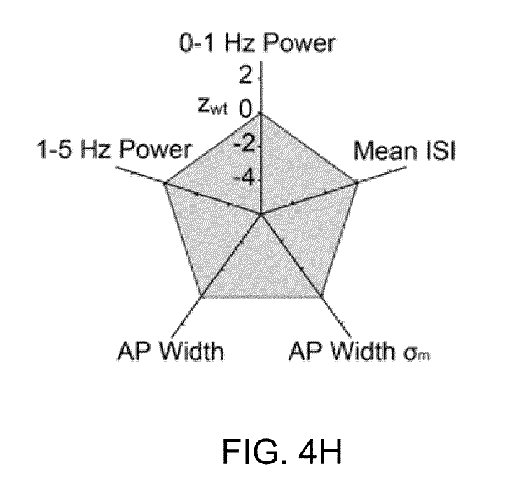

At least six specimens of each of the three varieties of C. elegans were characterized utilizing a microfluidic device. FIGS. 4A and 4B shows example microscopy images of the varieties during electrophysiology characterization. The microscopy images of FIGS. 4A and 4B are top down images having a similar perspective as the diagram shown in FIG. 3A.

Returning to FIG. 4A, the figure shows an example microscopy image of a C. elegans nematode (400) disposed in a microfluidic channel (410). The nematode (400) was placed into the microfluidic channel by the application of fluid pressure. Once placed in the channel (410), fluid pressure was applied to cause the nematode (400) to be positioned next to a horizontal electrode as shown in FIG. 4B.

FIG. 4B shows an example microscopy image of a C. elegans nematode (400) disposed proximate to a horizontal electrode (415). By causing the nematode (400) to be disposed proximate the horizontal electrode (415), the horizontal electrode (415) may make direct contact (420) with the nematode (400) and thereby provide direct contact to perform electrophysiology measurements.

Each of the varieties of nematode were subjected to electrophysiology characterization by the process of positioning each nematode shown in FIGS. 4A and B. When positioned, e.g., placed in direct contact with the horizontal electrode, electrophysiology measurements of each nematode were performed by instrumentation connected to the horizontal electrode.

FIG. 4C shows examples of the raw electrophysiology measurements of two example specimens of nematodes on a first day and a second day. The first and second curves show raw electrophysiology measurements of a nematode of the first variety taken on its first and second day of life, respectively. The third and fourth curves show raw electrophysiology measurements of a nematode of the second variety taken on its first and second day of life, respectively.

Based on the electrophysiology measurements of each nematode, averaged phenotypic maps were generated as shown in FIGS. 4D-4G. In the phenotypic maps shown in FIGS. 4D-4G, the statistical electrophysiology behavior of each variety of nematode was normalized to that of the wild type nematode, e.g., the light pentagram indicates the behavior of the wile type nematode and the dark overlaid pentagram indicates the behavior of the variety of nematode relative to the wild type nematode. Starting at 12 o'clock and following clockwise, each axis of the pentagrams indicate the fraction of the electrophysiology measurement having a frequency component of 1-2 Hz, the mean inter-spike interval, the standard deviation of all of the action potential widths, the average width of all of the action potential widths, and the fraction of the electrophysiology measurement having a frequency component of 1-5 Hz, respectively. An example legend is shown in FIG. 4H.

FIG. 4D shows a phenotypic map of the first variety of nematodes on the first day of life. As seen from FIG. 4D, the phenotypic map of the first variety of nematodes diverges from the map of the wild type map, e.g., the dark area is of a different shape than the light area.

FIG. 4E shows a phenotypic map of the first variety of nematodes on the second day of life. As seen from FIG. 4E, the phenotypic map of the first variety of nematodes has further diverged from the map of the wild type map by the second day of life.

FIG. 4F shows a phenotypic map of the second variety of nematodes on the first day of life. As seen from FIG. 4F, the phenotypic map of the second variety of nematodes diverges from the map of the wild type map along a number of axis.

FIG. 4G shows a phenotypic map of the second variety of nematodes on the second day of life. As seen from FIG. 4G, the phenotypic map of the second variety of nematodes has substantially diverged from the map of the wild type map by the second day of life. In particular, the first and second axis clock wise from the twelve o'clock position show very significant divergence.

As seen from FIGS. 4E and 4G, each phenotypic map illustrates substantially different electrophysiology behavior because the shapes of the phenotypic maps are different. Thus, the method described utilizing a microfluidic device may diagnostically evaluate conditions, e.g., ALD and PD, that have similar symptoms and distinguish between the conditions.

### EXAMPLE 2

In the following example, a microfluidic device including a horizontal electrode is utilized to evaluate the relief of symptoms of PD by a candidate drug. Specifically, the relief of symptoms of PD is evaluated by treating the second variety of nematodes discussed in Example 1.

As discussed with respect to Example 1, the second variety of nematodes may include gene expression that causes the second variety of nematodes that causes electrophysiology characterization of the second variety of nematodes to substantially differ from the wild type, as shown in FIGS. 4F and 4G.

To determine the relief of symptoms afforded by the candidate drug, a first portion of the second variety of nematodes was treated with the drug iodochlorhydroxyquin and a second portion of the second variety was not treated. Following the treatment, each portion of the second variety of nematodes were characterized by electrophysiology measurements as described with respect to FIGS. 4A and 4B. In other words, the portions of the second variety of nematodes were moved within the microfluidic device and thereby pressed against a horizontal electrode, disposed on a wall of the microfluidic device.

When placed against the horizontal electrode, electrophysiology measurements on the first and second portion of the second variety of nematodes. Phenotypic maps of the first and second portions of the second variety of nematodes were computed.

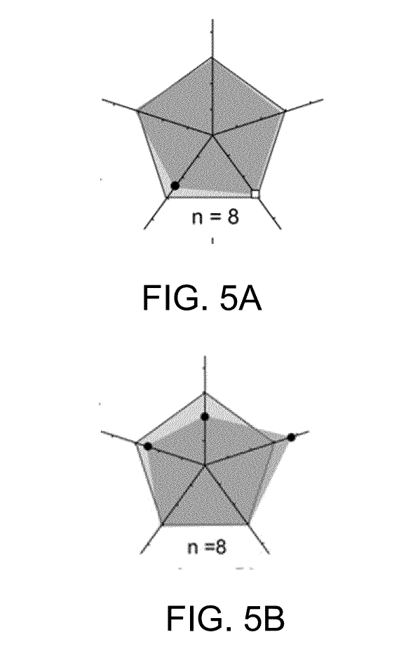

FIG. 5A shows a phenotypic map of the first portion of the second variety of nematodes on the second day of life of the nematodes. As noted above, the first portion was treated with iodochlorhydroxyquin. FIG. 5A shows that the application of iodochlorhydroxyquin reduced the divergence of the first portion of the second variety from the wild type, as indicated by the dark are of the pentagram more closely matching the light pentagram when compared to FIG. 4G.

FIG. 5B shows a phenotypic map of the second portion of the second variety of nematodes on the second day of life of the nematodes. As noted above, the second portion was not treated and thereby is a control group. FIG. 5B shows that without the application of iodochlorhydroxyquin, the electrophysiology characteristics of the second portion of the second variety of nematodes exhibits behavior indicative of the onset of symptoms of PD.

Thus, by performing electrophysiology characterization of the first portion of the second variety of nematodes that was treated with a drug and performing electrophysiology characterization of the second portion of the second variety of nematodes that was not treated, e.g., a control group, the effect of a candidate drug may be determined.

### EXAMPLE 3

In the following example, a microfluidic device including a horizontal electrode is utilized to purify cells on the basis of their electrophysiology characteristics. The microfluidic device is a part of a system as shown in FIG. 2A.

A supply of cells is provided to a supply control unit. The supply of cells may be, for example, cells dispersed in a buffer medium. The supply control unit feeds individual cells to a first end of the microfluidic device in response to commands received from the system controller. The supply control unit may feed the cells to the first end of the microfluidic device by utilizing fluid flow to position the cell.

Once a cell is supplied to the microfluidic device, fluid pressure is applied to the cell that causes the cell to traverse along the length of the microfluidic device to a region that is being monitored by an inspection system. The inspection system may be, for example, a microscope connected to a computer system. The computer system may include a machine vision system that recognizes the cell once it traverses to the monitored region.

Upon recognition of the cell, the inspection system may communicate with the system controller to traverse the cell proximate to a horizontal electrode disposed on a wall of the microfluidic device. Once the cell is proximate the horizontal electrode, the inspection system may notify the system controller.

In response to the notification, the system controller may activate a fluid control system that causes fluid to flow into one or more sub-channels and thereby cause the cell to traverse towards the horizontal electrode. Traversing towards the horizontal electrode may cause the cell to be placed in direct contact with the horizontal electrode and thereby enable electrophysiology measurements to be performed on the cell by a measurement control system connected to the horizontal electrode.

After the cell is characterized, a phenotypic map of the cell may be generated by the system controller and compared to a desired phenotypic map stored on the system controller. Based on the comparison, the cell may be sorted to a first tank if the phenotypic map of the cell meets the desired phenotypic map or the cell may be sorted to a second tank if the phenotypic map of the cell does not meet the desired phenotypic map.

Thus, by sorting the cells utilizing a microfluidic device including a horizontal electrode to perform electrophysiology characterization of the cells, a cell population having a desired phenotypic map may be produced.

### EXAMPLE 4

A microfluidic device in accordance with embodiments of the invention may be generated by forming a thin metal film made of on top surface of a glass wafer. The thin metal film may be formed by, for example, depositing metal by electron-beam evaporation. The thin metal film may be, for example, Titanium, Germanium, or Aluminum.

A photoresist may be spin-coated and cured on the thin metal layer. Horizontal electrodes may be formed by depositing a second metal layer on the photoresist layer and patterning the second metal layer to have a shape of a horizontal electrode. The second metal layer may be, for example, platinum. The second layer may be formed by, for example, depositing the metal by sputtering. The second layer may be patterned by, for example, depositing photoresist, processing the photoresist by photolithography, and performing a liftoff process to form the horizontal electrodes.

A second layer of thin cured photoresist may be deposited on the horizontal electrodes to electrically insulate the horizontal electrodes. The second layer of thin cured photoresist may have a thickness of, for example, 500 nm.

A sacrificial layer may be deposited above a portion of the glass slide at the location of where one or more microfluidic channels will be formed. At least a portion of one microfluidic substantially intersects with the horizontal electrode. The sacrificial layer may be formed from, for example, a polydimethylglutarimide based photo resist. The sacrificial layer may be patterned by photolithography to define the microfluidic channels. The sacrificial layer may have a thickness of 2 μm.

A third layer of photoresist may be deposited and patterned to define the microfluidic channels. The third layer may be, for example, an epoxy-based negative photoresist. The third layer may have a thickness of 25 μm.

Reactive Ion Etch may be performed to etch the microfluidic channels and suspend the horizontal electrodes. Reactive ion etching may also remove a portion of the first metal layer and thereby enable fluorescence microscopy and optical stimulation without interference from autofluorescence or photocurrent.

Once the microfluidic channels are formed, the sacrificial layer may be removed.

The sacrificial layer may be removed by, for example, wet etching. Removing the sacrificial layer may open the sub-channels shown in, for example, FIG. 2A.

### EXAMPLE 5

A microfluidic device in accordance with embodiments of the invention may be generated by forming a metal film made of on top surface of a 300 nm silicon dioxide (SiO2) film on a silicon (Si) wafer. The metal layer may be, for example, platinum. The metal layer may have a thickness of 60 μm. Other metal types and thicknesses may be used depending on the type of target.

Horizontal electrodes may be formed from the metal layer. The horizontal electrodes may be formed by depositing a photo resist, patterning the photo resist, and performing a left-off process. The electrodes may have a patterned with of 2-4 μm.

A dielectric layer may be deposited on the horizontal electrodes. The dielectric layer may be silicon dioxide. The dielectric layer may have a thickness of 200 nm. The dielectric layer may be deposited by electron-beam evaporation. The dielectric layer may be an insulating layer that insulates the horizontal electrodes.

Microfluidic channels may be formed within the wafer and silicon dioxide layers. At least a portion of one microfluidic channel may intersect with a horizontal electrode. The microfluidic channels may be formed by depositing a layer of photoresist, developing the photoresist, and etching to remove portions of the wafer and silicon dioxide layers.

The etching process may be a multistep etching process including a first step of reactive ion etching to remove portions of the silicon dioxide layer and a second step of cryogenic etching to remove portions of the wafer. The etching process may remove, for example, 25 μm of the wafer. The etching process may be partially isotropic and thereby remove portions of the wafer disposed below the horizontal electrodes. Removing a portion of the wafer disposed below the horizontal electrodes may suspend the horizontal electrodes within a microfluidic channel.

Thus, examples 4 and 5 show embodiments of the invention that may be utilized to characterize different types of targets.

Embodiments of the invention may accelerate genetic engineering of transmembrane proteins like ion channels and receptors can be used to alter cell physiology and activity. For example, the light-gated ion channel rhodopsin has been mutated in a variety of ways to produce larger ion currents, faster kinetics, and different activation spectra. To create these mutant variants many candidate mutants must be tested. Embodiments of the disclosure may greatly accelerate the process by rapidly testing the electrophysiological responses of cells expressing mutant variants and then collecting the cells that express mutant variants with improved properties.

Embodiments of the invention may enable rapid cell sorting based on their response to any number of potential agonists (optical, mechanical, thermal, pharmacological, etc.) to identify the sub sets of cells that express specific ion channels and receptors. Once these cells have been isolated, analysis of their gene expression may reveal the ion channels/receptors uniquely expressed in the isolated population.

Embodiments of the invention may accelerate the throughput of electrophysiology and may make rapid electrophysiology a viable metric for cell classification. Many diseases affect specific cell types making it critical to identify and classify groups of cells according to their similarities. Currently, cell type classifications are based primarily on morphology, gene expression, and to a lesser extent, electrophysiology. Electrophysiology is a powerful classifier since it can group cells based on their behavior rather than static properties like gene expression or morphology. Nevertheless, traditional electrophysiology is rarely used to classify cells because it is difficult to test and sort cells using current electrophysiology techniques.

Embodiments of the invention may enable electrophysiology measurements on a cell by cell basis of a population of cells exposed to a drug. Due to the diversity of ion channels, receptors, and cell types, characterizing a single cell via electrophysiology measurements does not properly capture the response of a population of cells exposed to a drug. Thus, the automated method of performing electrophysiology measurement may enable all cells of a population exposed to a drug to be screened, and therein the population response may be determined.

Embodiments of the invention may enable High-throughput and long small-organism electrophysiology. Small organisms reproduce very quickly and genetic characteristics of these organisms may be manipulated by controlling the reproduction cycle. High-throughput electrophysiology measurement techniques are needed to characterize these organisms to make determinations regarding manipulating the reproduction cycle of the population.

While the invention has been described above with respect to a limited number of embodiments, those skilled in the art, having the benefit of this disclosure, will appreciate that other embodiments can be devised which do not depart from the scope of the invention as disclosed herein. Accordingly, the scope of the invention should be limited only by the attached claims.

What is claimed is:

1. An electrophysiology device, comprising:
a first microfluidic channel comprising a side wall; and
an electrode, disposed on the side wall.

2. The electrophysiology device of claim 1, further comprising:
a second microfluidic channel,
wherein the first microfluidic channel intersects the second microfluidic channel,
wherein the intersection is proximate to the electrode.

3. The electrophysiology device of claim 1, wherein the first microfluidic channel further comprises a top side that is optically transparent.

4. The electrophysiology device of claim 1, wherein the electrode is attached to the side wall.

5. The electrophysiology device of claim 4, wherein a portion of the electrode extends into the first microfluidic channel, wherein the portion of the electrode is suspended in the first microfluidic channel.

6. The electrophysiology device of claim 1, wherein the electrode is a direct contact electrode.

7. The electrophysiology device of claim 1, wherein the electrode has a rectangular cross section.

8. The electrophysiology device of claim 1, wherein the electrode has a circular cross section.

9. The electrophysiology device of claim 1, wherein the electrode is a tubular structure.

10. The electrophysiology device of claim 1, wherein the electrode has the shape of a conic section.

11. The electrophysiology device of claim 10, wherein the conic section is a truncated cone.

12. The electrophysiology device of claim 11, wherein the conic section comprises a hollow portion.

13. The electrophysiology device of claim 1, wherein the electrode comprises:
a dielectric layer disposed on a portion of the electrode.

14. The electrophysiology device of claim 13, wherein the dielectric layer prevents contact between the portion of the electrode and a target when the target is disposed proximate the electrode.

15. The electrophysiology device of claim 1, wherein the side wall comprises:
a lower portion; and
an upper portion,
wherein a first portion of the electrode is disposed between the lower portion and the upper portion;
wherein a second portion of the electrode extends from the side wall.

16. The electrophysiology device of claim 1, further comprising:
an inspection system configured to monitor a target disposed within the first microfluidic channel; and
a system controller configured to position the target based on the monitoring by the inspection system.

17. The electrophysiology device of claim 16, wherein the system controller positions the target by generating a fluid flow within the first microfluidic channel.

18. A method of performing an electrophysiology measurement, comprising:
positioning, using a first fluid pressure, a target at a location, wherein the location is proximate an electrode;
generating, using a second fluid pressure, direct contact between the target and the electrode;
obtaining an electrophysiology measurement of the target while the target is in direct contact with the electrode; and
sorting the target based on the electrophysiology measurement.

19. The method of claim 18, further comprising:
generating a phenotypic map of the target based on the electrophysiology measurement; and
selecting a sorting tank based on a difference between the phenotypic map and a reference phenotypic map.

20. The method of claim 18, wherein the electrode is suspended within a microfluidic channel.
